(19) United States
(12) Patent Application Publication
(10) Pub. No.: US 2010/0267611 A1
(43) Pub. Date: Oct. 21, 2010

(54) TREATMENT OF METABOLIC DISEASE

(75) Inventor: Jeffrey Cohn, New South Wales (AU)

Correspondence Address: SCHWEGMAN, LUNDBERG & WOESSNER, P.A., P.O. BOX 2938, MINNEAPOLIS, MN, 55402, US

(73) Assignee: The University of Sydney, New South Wales (AU)

(21) Appl. No.: 12/740,747

(22) PCT Filed: Oct. 31, 2008
(86) PCT No.: PCT/AU2008/001622

§ 371 Date: Apr. 30, 2010

(30) Foreign Application Priority Data
Oct. 31, 2007 (AU) 2007905974

(51) Int. Cl. A61K 38/00; A61K 31/685; A61K 31/7028; A61P 3/00; A23J 7/00

(52) U.S. Cl. 514/1.1; 514/77; 514/25; 426/648

(57) ABSTRACT

The invention relates to metabolic disease, including non alcoholic fatty liver disease, to risk factors associated with same and to phospholipid-containing compositions.

### FIELD OF THE INVENTION

The invention relates to metabolic disease, including non alcoholic fatty liver disease, to risk factors associated with same and to phospholipid-containing compositions.

### BACKGROUND OF THE INVENTION

Many individuals having active metabolic disease such as non alcoholic fatty liver disease (NAFLD) tend to have accumulated a higher than normal amount of cholesterol and/or triglycerides in plasma or cells. These amounts of cholesterol and triglyceride are also observed in normal individuals receiving a high fat diet. Therefore a high fat diet, particularly one containing large amounts of saturated fat is widely recognised as being a significant risk factor for metabolic disease. The risk of metabolic disease becomes greater when individuals receiving a high fat diet have an excessive waist circumference (>102 cm for men, >88 cm for women) and/or non optimal blood pressure (≧130 mmHg systolic or ≧85 mmHg diastolic) and/or impaired fasting glucose (≧110 mg/dl).

The effect of dietary phospholipid on serum lipoproteins has been extensively studied in humans and experimental animals. Most of these studies have assessed polyunsaturated phospholipid in individuals having active disease. [Greten et al. 1980 “The effect of polyunsaturated phosphatidylcholine on plasma lipids and fecal sterol excretion” Atherosclerosis 36:81-88; Beil & Grundy 1980 “Studies on plasma lipoproteins during absorption of exogenous lecithin in man”. J. Lipid Res. 21: 525-536; Childs et al. 1981 “The contrasting effect of dietary soya lecithin product and corn oil on lipoprotein lipids in normolipdemic and familial hypercholesterolemic subjects” Atherosclerosis 38: 217-288; Zierenberg & Grudny 1982 “Intestinal absorption of polyenephosphatidylcholine in man”. J. Lipid Res. 23: 1136-1142; Simonsson et al. 1982 “Postprandial effects of dietary phosphatidylcholine on plasma lipoproteins in man.” Am. J. Clin. Nutr. 35: 36-41; Samochowiec et al. 1976 “Investigation in experimental atherosclerosis. Part 1. The effect of phosphatidylcholine one experimental atherosclerosis in white rats”. Atherosclerosis 23: 305-317; Rosseneu et al. 1979 Influence or oral polyunsaturated and saturated phospholipid treatment on the lipid composition and fatty acid profile of chimpanzee lipoproteins. Atherosclerosis 32: 141-153; Wong et al. 1980 “Lecithin influence on hyperlipemia in rhesus monkeys”. Lipids 15: 428-433; Clark et al. 1981 “Effect of lecithin ingestion on plasma and lymph lipoproteins of normo- and hyperlipemic rats”. Am. J. Physiol 241 422-G430; O'Mullane & Hawthorne 1982. “A comparison of the effects of feeding linoleic acid-rich lecithin or corn oil on cholesterol absorption and metabolism in the rats”. Atherosclerosis 45: 81-90].

More recent studies have shown that replacement of a portion of dietary fats with phospholipid can alleviate conditions associated with steatosis and hepatomegaly as commonly observed in individuals having NAFLD. In one study, 25% of dietary fat was replaced with phospholipid before triglyceride synthesis and enhancement of fatty acid β oxidation was observed in a model of fatty liver disease induced by orotic acid [Buang et al. 2005 Dietary phosphatidylcholine alleviateds fatty liver induced by orotic acid Nutrion 21: 867-873]. More recent studies have also focussed on the role of polyunsaturated phospholipid in the attenuation of alcoholic fatty liver diseases in non human primates [Navder et al. 1997 “Polvenylphosphatidylcholine decrease alcoholic hyperlipidemia without affecting the alcohol-induced rise of HDL-cholesterol”. Life Sci 61: 1907-14; Lieber et al. 1990 “Attentuation of alcohol-induced hepatic fibrosis by polyunsaturated lecithin” Hepatology 12:1390-8].

In summary of the above, the studies to date in relation to the affect of dietary phospholipids on accumulation of cholesterol and trigylceride have generally assessed:
cases of active disease;
unsaturated forms of dietary phospholipid;
large amounts of phospholipid—about 20 to 30% of total dietary fats;
replacement of dietary fats with phospholipid.

There remains a need to treat individuals having metabolic disease characterised by an accumulation of cell or plasma-associated cholesterol and/or triglyceride.

There also remains a need to reduce the accumulation of cell or plasma-associated cholesterol and/or triglyceride in individuals at risk for metabolic disease, especially individuals receiving a high fat diet.

### SUMMARY OF THE INVENTION

In certain embodiments there is provided a method for preventing the accumulation of plasma or cell-associated triglyceride and/or cholesterol in an individual having one or more risk factors for a metabolic disease. The method includes the step of providing an individual having one or more risk factors for a metabolic disease with a composition including an amount of phospholipid effective for lowering triglyceride and/or cholesterol in the individual.

In other embodiments there is provided a method of treating an individual having a disease or condition characterised by an accumulation of plasma or cell-associated triglyceride or cholesterol. The method includes the step of providing an individual having a disease or condition characterised by an accumulation of plasma or cell-associated triglyceride or cholesterol with a composition including an amount of phospholipid effective for lowering triglyceride and/or cholesterol in the individual.

In further embodiments there is provided a use of a composition including an amount of phospholipid effective for lowering triglyceride and/or cholesterol in an individual for treating an individual at risk of or having a disease or condition characterised by an accumulation of plasma or cell-associated triglyceride or cholesterol.

In further embodiments there is provided a use of a phospholipid to manufacture a composition in the form of a medicament, dietary supplement, or functional food that has an amount of phospholipid effective for lowering triglyceride and/or cholesterol in an individual, the composition for treating an individual at risk of or having a disease or condition characterised by an accumulation of plasma or cell-associated triglyceride or cholesterol.

In further embodiments there is provided a composition in the form of a nutraceutical, pharmaceutical, food or dietary supplement, the composition having an amount of phospholipid effective for lowering triglyceride and/or cholesterol in an individual.

In further embodiments there is provided a functional food including an amount of phospholipid effective for lowering triglyceride and/or cholesterol in an individual.

### BRIEF DESCRIPTION OF THE DRAWINGS

FIG. 1. Historical appearance of liver sections from N: a normal chow-fed mice; NPL: a normal chow-fed mice supplemented with composition; HF: a high-fat fed mice; HFPL: a high-fat fed ice supplemented with composition. Left-hand panels show sections stained with hematoxylin and eosin. Right-hand panels show sections stained with Oli-Red-O. Lipid accumulation in the liver of HF-fed mice was very evident due to the presence of circular lipid droplets in the H&E stained sections and intense red staining in Oil-Red-O stained sections. Circular lipid droplets and red staining were significantly reduced in sections from HFPL-fed animals.

FIG. 2. Total lipid in the liver of mice fed a normal chow or high-fat diet with or without the addition of phospholipid-rich dairy milk extract. Top panels shows results obtained gravitometrically expressed as grams of lipid per 100 grams of liver; bottom panel shows results expressed as grams of lipid per whole liver. Results represent means±SE. Mice were fed diets for 8 weeks. Values in the same row sharing a common letter are significantly different (P<0.05) by ANOVA followed by Turkey's multiple comparison test. N: normal chow-fed mice; NPL: normal chow-fed mice supplemented with composition; HF: high-fat fed mice; HFPL: high-fat fed mice supplemented with composition (n=10 per group).

FIG. 3. Serum glucose in mice fed chow or high-fat diet with or without supplementation with the phospholipid containing composition. Significantly different N v NPL or HF v HFPL: * P<0.05, ** P<0.01.

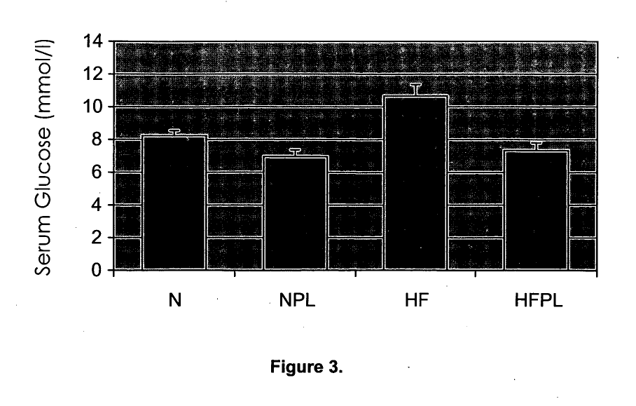

### DETAILED DESCRIPTION OF THE EMBODIMENTS

In certain embodiments there is provided a use of a composition including an amount of phospholipid effective for lowering triglyceride and/or cholesterol in an individual for treating an individual at risk of or having a disease or condition characterised by an accumulation of plasma or cell-associated triglyceride or cholesterol.

In certain embodiments the phospholipids having a higher relative abundance in the composition are those having a saturated fatty acyl chain. In one embodiment, at least 60% of the phospholipids in the composition have a saturated fatty acyl chain. In another embodiment, phospholipids having a monounsaturated fatty acyl chain constitute no more than about 30% of phospholipids in the composition. In other embodiments, the phospholipids having a polyunsaturated fatty acyl chain constitute no more than about 10% of phospholipids in the composition. One example of a composition useful in the above described method is one having a ratio of about 6 to 3 to 1 parts of saturated, monounsaturated and polyunsaturated phospholipids respectively. In another example, about 70% of phospholipids in the composition have a saturated fatty acyl chain.

The composition may further include a triglyceride in a ratio of about 5 parts phospholipid to 1 part triglyceride.

In one embodiment, at least 20% of the fatty acyl chains of the composition have a length of no more than 14 carbon atoms. About 30% of the fatty acyl chains of the composition may have length of 16 carbon atoms. At least 50% of the fatty acyl chains of the composition may have a length of no more than 16 carbon atoms. About 35% of fatty acyl chains of the composition may have a length of 18 carbon atoms or greater.

The phospholipid may be selected from the group consisting of phosphatidyl ethanolamine (PE), phosphatidyl choline (PC), phosphatidyl serine (PS), phosphatidyl inositol (PI) and sphingomyelin.

In one embodiment, the composition may include PE in an amount of about 30% of total phospholipids. The composition may include PC in an amount of about 30% of total phospholipids. The composition may include PS in an amount of about 15% of total phospholipids. The composition may include PI in an amount of about 10% of total phospholipids. The composition may include sphingomyelin in an amount of about 25% of total phospholipids.

The composition may include a non lipid component in an amount of about 45% by weight of the composition. The non lipid component may include protein in an amount of about 25% by weight of the non lipid component. The protein may include casein although other proteins such as soy protein or gluten could be used instead.

The non lipid component may include carbohydrate in an amount of about 1% by weight of the non lipid component. Examples of carbohydrates include lactose, glucose, starch.

In one embodiment, the composition for use in the methods described herein is produced by admixing purified sources of phospholipid, tryglycerides and non fat components.

Purified phospholipids may be obtained from Lipoid GmbH, Frigenstr. 4, D-67065, Ludwigshafen, Germany (http://www.lipoid.com/contacts/index.html)
Triglycerides may be obtained from Sigma-Aldrich (http://www.sigmaaldrich.com/Area of Interest/Asia Pacific Rim/Australia.html)
Non fat components may be obtained from Sigma-Aldrich (http://www.sigmaaldrich.com/Area of Interest/Asia Pacific Rim/Australia.html)

In other embodiments, one or more of the phospholipids, triglycerides and non fat components may be obtained from a plant and/or animal source. For example, phospholipids and triglycerides may be obtained from tallow or soy and non fat components provided in the form of soy protein.

In still further embodiments, the composition is provided by obtaining non fat component, such as hydrolysed protein from a plant or animal source and adding purified phospholipids and/or triglycerides to it in amounts as described above. For example purified phospholipids and/or triglycerides may be added to soy protein to provide a composition having a fat component of about 60% of the total composition weight, the fat component having a ratio of about 6 to 3 to 1 parts of unsaturated, monounsaturated and polyunsaturated phospholipids respectively.

In other embodiments, the composition is provided by obtaining phospholipids and triglycerides from a plant or animal source and adding purified protein or protein extract to it. For example, soy bean oil may be provided with an amount of saturated and monounsaturated phospholipids and triglycerides to provide a composition having a ratio of about 6 to 3 to 1 parts of saturated, monounsaturated and polyunsaturated phospholipids respectively. An extract of soy protein may also be added to provide the composition with an amount of non fat component of about 40 to 45% of the total composition weight.

In one embodiment the phospholipids are dairy phospholipids such as phospholipids obtained from a mammalian milk, especially bovine milk, or from a fraction, extract or product thereof.

The composition for use in the methods described above may be provided for use as a supplement in the form of a nutraceutical, food or dietary supplement. In these embodiments, the composition is used to minimise the accumulation of plasma or cell-associated triglyceride and/or cholesterol in an individual having one or more risk factors for a metabolic disease. For example a nutraceutical, food or dietary supplement could be used to supplement a high fat diet, especially a high fat diet having large amounts of saturated fat.

In an alternative embodiment a functional food is provided including an amount of phospholipid effective for lowering triglyceride and/or cholesterol in an individual. The functional food is characterised in that a portion of fat that would otherwise form an ingredient in the manufacture of a given food is replaced with a composition as described above, or an amount of phospholipid effective for lowering triglyceride and/or cholesterol in an individual as described herein.

In one embodiment the individual to receive the composition disclosed herein is one having one or more risk factors for a metabolic disease. Typically the risk factor is a high fat diet, especially a diet having large amounts of saturated fat. An example of a high fat diet is one that results in the accumulation of abnormal amounts of plasma cholesterol or triglyceride or otherwise results in disturbance of lipid metabolism (such as for example triglycerides >150 md/dl or low density lipoprotein >130 mg/dl cholesterol or total cholesterol >200 mg/dl or high density lipoprotein cholesterol <60 mg/dl).

The individual may additionally or alternatively have other risk factors for metabolic disease including:
an excessive waist circumference (>102 cm for men, >88 cm for women);
an increased body mass index (greater than 25 kg/m²);
non optimal blood pressure (≧130 mmHg systolic or ≧85 mmHg diastolic);
impaired fasting glucose (fasting plasma glucose values of 100 to 125 mg per dL (5.6 to 6.9 mmol/l));
impaired glucose tolerance (two-hour 75-g oral glucose tolerance test values of 140 to 199 mg per dL (7.8 to 11.0 mmol/l));
insulin resistance (fasting blood insulin level greater than 20 mcU/mL);
first-degree relatives who are suffering or have suffered from metabolic disease, including a genetic disease such as hyperlipidemia.

Examples of individuals having these risk factors include those at increased risk of developing new onset diabetes, such as those with a pre-diabetic state, for example metabolic syndrome or syndrome X. Metabolic syndrome or syndrome X is defined here on the basis of NCEP ATP III criteria, which are the presence of three or more of the following factors: 1) increased waist circumference (>102 cm [>40 in] for men, >88 cm [>35 in] for women); 2) elevated triglycerides (≧150 mg/dl); 3) low HDL cholesterol (<40 mg/dl in men, <50 mg/dl in women); 4) non-optimal blood pressure (≧130 mmHg systolic or ≧85 mmHg diastolic); and 5) impaired fasting glucose (≧110 mg/dl).

In the above embodiments, the phospholipids in the composition are provided as a supplement to the dietary fats. For example the composition may be provided in the form of a nutraceutical, food or dietary supplement or functional food.

In other embodiments, the individual to receive the composition disclosed herein is one having a disease or condition characterised by an accumulation of plasma or cell-associated triglyceride or cholesterol. The accumulation may stem from overproduction or reduced catabolism of triglyceride and/or cholesterol.

The disease or condition is typically a metabolic disease including an disease or condition at an early onset stage or in an active stage.

The composition of the invention may be useful for preventing, delaying, slowing, arresting or treating diseases or conditions including neuropathy, nephropathy, retinopathy, chorioretinopathy, choroidal neovascularisation, retinal neovascularisation, macular degeneration, retinal detachment, glaucoma, cataract, microangiopathy, atherosclerosis, ischemic heart disease, ischemic cerebrovascular disease, stroke, peripheral arteriosclerosis, cerebral arteriosclerosis, coronary arteriosclerosis, hyperinsulinemia induced sensory disorder, obesity, heart failure, myocardial infarction, angina pectoris, cerebral infarction, chronic cardiomyopathy, cardiac fibrosis, renal disorders, glomerular nephritis, glomerulosclerosis, nephritic syndrome, hypertensive nephrosclerosis, terminal renal disorders, and diabetic cachexia.

In certain embodiments the individual to be treated is one selected for having early or active forms of non alcoholic fatty liver disease including hepatic steatosis and hepatomegaly.

In certain embodiments the individual to be treated is one having elevated levels of aspartate aminotransferase (AST) and alanine aminotransferase (ALT), especially elevated levels in hepatic tissue.

In the above described embodiments, the composition may be provided in the form of a pharmaceutical including highly purified and defined quantities of phospholipids in amounts described herein.

In certain embodiments, the composition including an amount of phospholipid effective for reducing triglyceride and/or cholesterol in an individual prevents, inhibits, reduces, or interferes with accumulation of triglyceride and/or cholesterol in plasma, cells such as hepatocytes or tissues or organs. In these embodiments, the composition reduces the amount or concentration of triglyceride and/or cholesterol in plasma to normal levels. Normal levels of triglycerides are generally less than 150 mg/dl. Normal levels of low density lipoprotein are generally less than 130 mg/di cholesterol. Normal levels of total cholesterol are generally less than 200 mg/dl. Normal levels of high density lipoprotein cholesterol are generally greater than 60 mg/dl.

The composition may prevent the accumulation of triglyceride and/or cholesterol by reducing intestinal absorption of triglyceride, cholesterol or bioacids. In other embodiments, accumulation is prevented by the phospholipids in the composition affecting hepatic metabolism leading to a reduction in triglyceride synthesis. In other embodiments, accumulation is prevented by the phospholipids in the composition affecting the metabolism of triglycerides in muscle tissue.

In certain embodiments, the composition including an amount of phospholipid effective for reducing triglyceride and/or cholesterol in an individual prevents or reduces the conversion of pyruvate to glucose in the individual.

In certain embodiments the composition is provided as a unit dosage form such as a capsule, pill, caplet or like product, each unit dosage form having an amount of phospholipid from about 0.5 g to 5 g, preferably 1 to 4 g or an amount within these ranges.

In certain embodiments the total amount to be provided per day is generally around 5 g or less. This may be provided by a once daily dose amount to the required amount per day, or in multiple doses to be taken at meal times such as 1.5 g at breakfast, lunch and dinner, or 2.5 g at least 2 of breakfast lunch and dinner. Amounts greater than 5 g may be required depending on the risk factors and/or the disease or condition that an individual may have.

In other embodiments, especially where the composition is provided in the form of a functional food, the phospholipids are provided in an amount of about 0.5 g to 5 g of phospholipid per 100 grams of food, preferably 1 to 4 g of phospholipid per 100 grams of food or an amount within these ranges.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Materials and Methods

### Animals and Diets

Six-week old male C57BL/6 mice were obtained from the Australian Resources Centre (Perth, Australia). Animals were housed in standard cages (5 animals per cage) at a constant temperature of 20° C. with a 12 h light/dark cycle. They were allowed ad libitum access to diet and water. After 1 week of acclimatization, they were divided into four groups (n=10 mice per group): 1) normal chow-fed group (N) that received normal mice pellets containing 4.6% by weight of fat; 2) normal chow-fed group supplemented with a 2.55% wt/wt composition having a relative amount of phospholipids (NPL); 3) high-fat fed group (HF) that received a diet containing 21% wt/wt butterfat and 0.15% cholesterol; and 4) high-fat fed group supplemented with a 2.55% wt/wt composition having a relative amount of phospholipids (HFPL). Food intake was recorded weekly and body weights were measured 3 times a week.

### Tissue Processing

Mice were fed diets for 8 weeks and after an overnight fast were exsanguinated by heart puncture under methoxyflurane anaesthesia. Blood was allowed to clot and serum was separated by ultracentrifugation (3,000 rpm, 10 min). Sera were aliquoted and stored frozen (−80° C.) until analysis. Livers were immediately excised, weighed and divided into smaller pieces for storage at −80° C. (for lipid analysis), in RNAlater® Solution (Albion, Austin, Tex.) (for gene expression analysis) or in 4% paraformaldehyde for histological analysis. Epididymal, inguinal and perirenal fat pads, as well as the quadriceps muscle from one leg, were excised and weighed. Liver samples were examined histologically after embedding in paraffin, sectioning, and staining with hematoxylin and eosin. Frozen sections were also stained with Oil-Red-O.

### Biochemical Analyses

Liver enzymes—alanine aminotransferase (ALT) and aspartate aminotransferase (AST)—were measured with colorimetric endpoint assays using commercial reagents (Teco Diagnostic, Anaheim, Calif.). Blood glucose was measured in whole blood with a glucose meter (Accu-Chek Integra, Roche Diagnostics). Serum insulin was measured by ELISA (Ultra Sensitive ELISA Kit, Crystal Chem Inc.). Serum triglyceride, cholesterol, and free fatty acid concentrations were measured by enzymatic methods, using GPO-PAP and CHOD-PAP (Roche Diagnostics) and Wako NEFA C (Wako Pure Chemicals, Osaka, Japan) kits, respectively. Serum phospholipid levels were measured enzymatically according to standard techniques. Serum HDL was separated by polyethylene glycol precipitation of apoB-containing lipoproteins (50p1 of polyethylene glycol (200 mg/ml) added to 50p1 of serum). Serum apoA-I concentration was measured by competitive ELISA, whereby goat anti-mouse apoA-I polyclonal antibody was used as the capture antibody, rabbit anti-mouse apoA-I polyclonal antibody was used for detection, and goat anti-rabbit IgG HRP conjugated was used for quantifcation (Biodesign International, Saco, Me.). Total liver lipids were determined gravitometrically after extraction by standard methods. Individual hepatic lipids were quantitated enzymatically (as described above) after solubilization in isopropanol.

### Results

The composition was administered to the NPL and HFPL groups. Almost half of the composition is composed of PL (i.e., 47.9%), which represented 84% of the total fat. The major PL components are PE, PC and SM, with smaller amounts of PS and PI. The remaining PL consists of lysophospholipids. The main fatty acids present are palmitic (C16:0), stearic (C18:0) and oleic (C18:1) acids. The S:M:P ratio of the composition is 60:30:10. About one-third of the composition is composed of protein. Since the aim of the present study was to investigate the effect of diets containing 1.2% PL by weight, the composition was added at a level of 2.5% by weight.

Addition of the composition did not affect the amount of food consumed per day. Mean food intake (±SE) for the N and NPL groups was 3.8±0.1 and 4.0±0.1 g/mouse/day, respectively. Mean food intake for the HF and HFPL groups was 3.3±0.2 and 3.2±0.3 g/mouse/day, respectively (measured during the second half of the study). The composition also did not affect total body weight gain, as shown in Table 1. Neither mean final body weights nor mean weight gains were significantly different (N v NPL or HF v HFPL), despite the fact that HF-fed animals gained significantly more weight than chow-fed animals.

However, the composition had a significant effect on liver weight in HF-fed mice (Table 1). Liver weights were 24±3% lower in HFPL-fed v HF-fed mice (P<0.001). N and NPL liver weights were not significantly different. Mean liver weight was 39±6% higher in the HF compared to the N group (P<0.001). This was not however the case for the HFPL compared to the N group (6±4%, P=0.17 ns). When expressed relative to total body weight, liver weight ratios in HF and HFPL groups were significantly higher and lower, respectively, compared to the N group (Table 1). Epididymal fat mass was similar in HF and HFPL animals (1634±77 v 1566±100 mg, respectively). Peroneal fat mass was also similar (421±23 v 401±31 mg). Inguinal fat mass was however higher in HF compared to HFPL animals (1409±92 v 1119±80 mg, P<0.05).

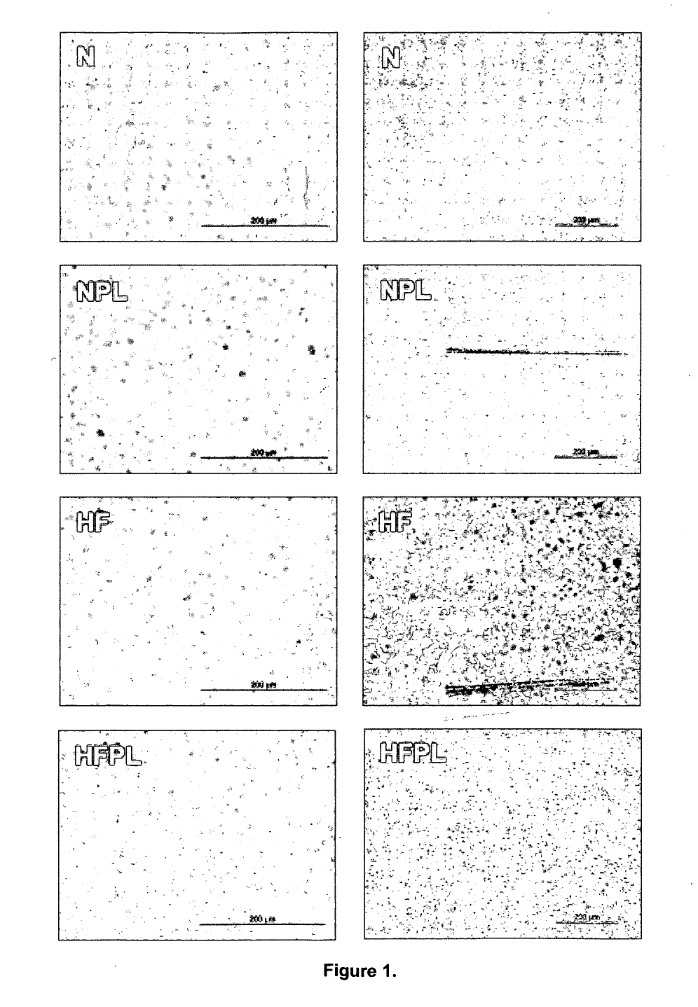

Livers from mice fed the four different diets were analyzed histologically and representative stained sections are shown in FIG. 1. No apparent differences were observed between liver sections from N and NPL mice, whether stained with H&E (lefthand panels) or oil-red-o (right-hand panels). H&E sections from HF animals revealed the presence of a large number of circular lipid droplets that stained red in oil-red-o treated sections. These lipid inclusions were clearly reduced in both size and number in livers of HFPL-treated animals.

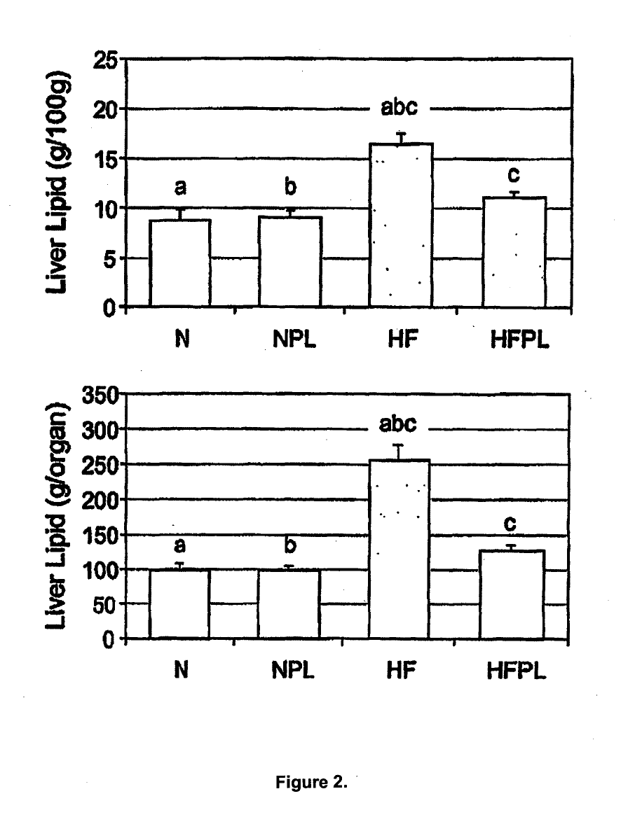

The beneficial effect of the composition on HF-induced hepatomegaly was associated with a significant reduction in total hepatic lipid content, expressed as grams of lipid per 100 grams of liver, or expressed as grams of lipid per whole liver (FIG. 2). Total liver content (in g/100 g) was 33±3% lower and (in g/organ) was 50±3% lower in HFPL compared to HF mice. HFPL-fed mice had liver lipid levels that were higher, though not significantly, than N-fed mice (i.e., 26±7% and 29±8%, for g/100 g and g/organ, respectively). No significant difference was observed between total liver lipid levels of N and NPL mice. As shown by the data in Table 2, measurement of liver triglyceride (TG), cholesterol (Chol) and phospholipid (PL) revealed that: 1) N and NPL mice had similar levels of liver TG, Chol and PL; 2) HF mice had significantly elevated levels of all 3 lipids compared to N mice (i.e., 2.8-fold (±0.3) increase in TG; 6.2-fold (±1.1) increase in Choi; and 1.5-fold (±0.1) increase in PL); 3) the addition of the composition to the diet of HF fed mice resulted in a significant decrease in hepatic TG and Chol, though not PL (i.e., decreases of 44±3%, 48±5% and 16±5%, resp.).

Liver enzymes, aspartate aminotransferase (AST) and alanine aminotransferase (ALT), were measured in serum as indicators of liver damage. Mean (±SE) ALT activities were 36.5±3.4, 37.6±2.1, 54.9±3.9 and 46.1±2.3 (U/l) for the N, NPL, HF, HFPL groups respectively. HF activities were significantly greater than those of N and NPL (P<0.05), and HFPL activities were less than those of HF (P=0.06). Mean (±SE) AST activities were 159±18, 175±11, 185±18 and 170±11 (U/l) for the N, NPL, HF, HFPL groups, respectively, which were not significantly different.

Plasma lipid and lipoprotein levels of mice fed the four diets are shown in Table 3. No significant differences were observed between N and NPL mice. Compared to N fed mice, HF mice had significantly elevated levels of serum TG, Chol, PL, non-HDL Chol, HDL Chol and apoA-I. HFPL mice in turn had lower levels of these parameters compared to HF-fed animals (i.e., TG, Chol and PL levels were reduced by 20±3%, 23±4%, and 21±4%, respectively). HDL Chol and apoA-I levels, which were elevated 2.0-fold and 1.7-fold in HF-fed mice, were reduced in HFPL-fed animals by 23±5% and 19±2%, respectively. Serum glucose levels in N and NPL mice were: 8.2±0.3 and 6.7±0.4 mmol/l (P<0.01) and in HF and HFPL mice were 9.0±0.6 and 6.2±0.4 mmol/l, (P<0.001). Serum insulin levels were not significantly different (pmol/l): N: 36.6±5.3, NPL: 58.3±7.1, HF: 49.7±6.1 and HFPL: 39.0±6.3, respectively.

### Discussion

Results of the present study demonstrate that the addition of the composition to the diet of chow-fed mice (representing 1.2 g PL per 100 g food) has little effect on metabolic parameters. However, when added to a high-fat diet containing 21% butterfat and 0.15% cholesterol, the composition resulted in a significant reduction in: a) liver weight, b) total liver lipid; c) liver triglyceride and cholesterol; and d) serum lipid levels. These data indicate that the composition has a beneficial effect on hepatomegaly, hepatic steatosis and elevated serum lipid levels in mice fed a high-fat diet.

Studies involving soybean and safflower phospholipids have consistently demonstrated that dietary vegetable-derived phospholipids containing unsaturated fatty acids can significantly lower cholesterol and triglyceride levels in both plasma and liver. A unique feature of the present study is the finding that phospholipids containing predominantly saturated fatty acids, can have a similar effect. Sixty percent of the fatty acids in the composition were saturated, 30% were monounsaturated, and only 10% were polyunsaturated. This compares to the S:M:P ratio of soybean phospholipids, reported to be 23:10:67, 24:8:68 or 28:11:71, and to that of safflower phospholipids, reported to be 28:65:22. These data support the concept that the lipid-lowering properties of dietary phospholipids are not dependent on the degree of saturation of their component fatty acids. This in turn implies that the base moiety and hence the type of phospholipids present is an important contributing factor.

The significant effect of the composition on liver weight and liver lipid content in high-fat fed mice suggests that it is to be expected to be of therapeutic benefit in humans with non-alcoholic fatty liver disease (NAFLD).

In conclusion, the present data indicates that the composition described herein has a beneficial effect on hepatomegaly, hepatic steatosis and elevated serum lipid levels in mice fed a high-fat diet.

TABLE 1
Body and liver weights of mice fed normal chow or a
high-fat diet with or without composition

| | N | NPL | HF | HFPL |
|---|---|---|---|---|
| Initial body wt. (g) | 21.3 ± 0.2 a | 21.1 t 0.4 b | 21.3 t 0.3 c | 21.3 ± 0.3 d |
| Final body wt. (g) | 29.2 f 0.5 ac | 30.7 ± 1.1 b | 34.3 t 0.5 ab | 33.4 ± 0.9 C |
| Wt. gain(g) | 7.9 ± 0.4 ac | 9.6 ± 0.8 bd | 13.0 ± 0.3 ab | 12.1 ± 0.$ cd |
| Liver wt. (g) | 1.13 t 0.02 a | 1.18 t 0.06 b | 1.57 ± 0.06 abc | 1.20 ± 0.04 c |
| Liver wt./body wt. (gi100 g) | 4.27 0.03 a | 4.18 t 0.07 b | 4.85 ± 0.14 ab | 3.80 ± 0.06 ab |

Results represent means ± SE.
Mice were fed diets for 8 weeks.
Values in the same row sharing a common letter are significantly different (P < 0.05) by ANOVA followed by Tukey's multiple comparison test.
N: normal chow-fed mice;
NPL: normal chow-fed mice supplemented with composition;
HF: high-fat fed mice;
HFPL: high-fat fed mice supplemented with composition of the invention (n = 10 per group).

TABLE 2
Liver lipid levels of mice fed normal chow or a high-fat
diet with or without added composition

| | N | NPL | HF | HFPL |
|---|---|---|---|---|
| Triglyceride (μmol/g) | 79.9 ± 12.6^{a} | 91.8 ± 7.7^{b} | 220.4 ± 21.6^{abc} | 124.0 ± 6.9^{c} |
| Cholesterol (μmol/g) | 6.4 ± 1.0^{a} | 6.5 ± 0.6^{b} | 39.7 ± 6.8^{ab} | 20.7 ± 2.0^{ab} |
| Phospholipid (μmol/g) | 21.8 ± 1.7^{a} | 24.0 ± 1.6^{b} | 33.6 ± 1.7^{ab} | 28.2 ± 1.6^{c} |

Results represent means ± SE. Mice were fed diets for 8 weeks.
Values in the same row sharing a common letter are significantly different (P < 0.05) by ANOVA followed by Turkey's multiple comparison test.
N: normal chow-fed mice;
NPL: normal chow-fed mice supplemented with composition;
HF: high-fat fed mice;
HFPL: high-fat fed mice supplemented with composition (n = 10 per group).

TABLE 3
Plasma lipid and lipoprotein levels in mice fed normal chow or a high-fat diet
with or without added composition.

| | N | NPL | HF | HFPL |
|---|---|---|---|---|
| Triglyceride (mmol/l) | 1.04 ± 0.09^{a} | 1.24 ± 0.11^{b} | 1.36 ± 0.06^{a} | 1.09 ± 0.04^{c} |
| Phospholipid (mmol/l) | 2.36 ± 0.08^{a} | 2.48 ± 0.04^{b} | 3.32 ± 0.11^{abc} | 2.61 ± 0.12^{c} |
| Cholesterol (mmol/l) | 1.82 ± 0.09^{a} | 1.95 ± 0.07^{b} | 4.61 ± 0.16^{abc} | 3.56 ± 0.17^{abc} |
| Non-HDL chol. (mmol/l) | 0.34 ± 0.08^{a} | 0.32 ± 0.03^{b} | 1.60 ± 0.08^{abc} | 1.12 ± 0.11^{abc} |
| HDL chol. (mmol/l) | 1.47 ± 0.05^{a} | 1.67 ± 0.03^{b} | 3.00 ± 0.11^{abc} | 2.44 ± 0.07^{abc} |
| ApoA-I (mg/dl) | 30.0 ± 1.3^{a} | 33.5 ± 1.1^{b} | 50.1 ± 2.0^{ab} | 38.7 ± 2.5^{a} |

Results represent means ± SE. Mice were fed diets for 8 weeks.
Values in the same row sharing a common letter are significantly different (P < 0.05) by ANOVA followed by Turkey's multiple comparison test.
N: normal chow-fed mice;
NPL: normal chow-fed mice supplemented with composition;
HF: high-fat fed mice;
HFPL: high-fat fed mice supplemented with composition (n = 10 per group).

Glucose levels were also significantly lower in phospholipid composition-supplemented mice at baseline. These data suggested that the composition was having an effect on hepatic glucose metabolism and/or hepatic glucose insulin sensitivity.

TABLE 4
Serum glucose concentration at time-points after intraperitoneal
injection of glucose in mice fed diets for 7 weeks.

| | Baseline | 15 min | 30 min | 60 min | 120 min |
|---|---|---|---|---|---|
| N | 7.6 ± 0.3 | 16.8 ± 0.9 | 16.9 ± 0.9 | 12.4 ± 0.7 | 8.4 ± 0.5 |
| NPL | 6.5 ± 0.4* | 15.7 ± 1.2 | 16.8 ± 1.2 | 13.5 ± 0.9 | 8.8 ± 0.5 |
| HF | 11.1 ± 1.0 | 18.4 ± 1.0 | 21.8 ± 1.3 | 17.7 ± 1.5 | 10.4 ± 0.8 |
| HFPL | 8.2 ± 0.6* | 19.9 ± 1.4 | 20.4 ± 1.1 | 17.8 ± 1.0 | 10.3 ± 0.4 |

Significantly different NPL v N and HFPL v HF:
*P < 0.05

Liver levels of glucose and glycogen were measured (Table 5.).

TABLE 5
Hepatic glucose and glycogen levels in mice fed diets for 7 weeks.

| | N | NPL | HF | HFPL |
|---|---|---|---|---|
| Glucose | 0.96 ± 0.13 | 0.69 ± 0.15 | 0.65 ± 0.11 | 0.26 ± 0.04** |
| (mg/g) | | | | |
| Glycogen | 0.18 ± 0.05 | 0.12 ± 0.03 | 0.05 ± 1.2 | 0.06 ± 0.01 |

Significantly different NPL v N and HFPL v HF: **P < 0.05

No significant differences were observed in liver glycogen levels.

Glucose was lower in phospholipid composition-supplemented v unsupplemented animals. This was statistically significant in HF-fed animals (P=0.09 in chow-fed mice).

In order to assess hepatic glucose production, we injected pyruvate intraperitoneally. Results in Table 6 indicate that conversion of pyruvate to glucose is reduced in phoshpolipid composition-supplemented mice.

RT-PCR analysis (measurement of liver mRNA levels) has shown that the hepatic expression of genes regulating gluconeogenesis is reduced in phoshpolipid composition-supplemented mice, supporting the concept that dietary phospholipid affects hepatic glucose production.

TABLE 6
Serum glucose concentration at time-points after intraperitoneal
injection of pyruvate in mice fed diets for 8 weeks

| | Baseline | 15 min | 30 min | 60 min | 120 min |
|---|---|---|---|---|---|
| N | 7.4 ± 0.2 | 10.8 ± 0.4 | 13.3 ± 0.6 | 11.1 ± 0.7 | 7.0 ± 0.4 |
| NPL | 6.1 ± 0.3** | 9.5 ± 0.4* | 11.4 ± 0.6* | 11.5 ± 0.7 | 7.5 ± 0.3 |
| HF | 7.5 ± 0.3 | 11.3 ± 0.6 | 11.9 ± 0.7 | 11.3 ± 0.7 | 8.1 ± 0.6 |
| HFPL | 7.0 ± 0.4 | 9.4 ± 0.5* | 11.1 ± 0.6 | 11.2 ± 0.6 | 8.3 ± 0.3 |

Significantly different NPL v N and HFPL v HF:
*P < 0.05,
**P < 0.01

1. Use of a phospholipid in the manufacture of a composition for minimising the accumulation of plasma or cell-associated triglyceride or cholesterol in an individual having one or more risk factors for metabolic disease, or for treating an individual having a disease or condition characterised by an accumulation of plasma or cell-associated triglyceride or cholesterol, wherein the composition includes unsaturated, monounsaturated and polyunsaturated phospholipids in a ratio of about 6 to 3 to 1 respectively.

2.-3. (canceled)

4. The use according to claim 1 wherein the composition further comprises a triglyceride in a ratio of about 5 parts phospholipid to 1 part triglyceride.

5. The use according to claim 1 wherein at least 20% of the fatty acyl chains of the phospholipids have a length of no more than 14 carbon atoms.

6. The use according to claim 1 wherein at least 30% of the fatty acyl chains of the phospholipids have a length of no more than 16 carbon atoms.

7. The use according to claim 1 wherein at least 50% of the fatty acyl chains of the phospholipids have a length of no more than 18 carbon atoms.

8. The use according to claim 1 wherein no more than about 70% of the fatty acyl chains of the phospholipids are saturated.

9. The use according to claim 1 wherein the composition includes PE in an amount of about 30% of total phospholipids.

10. The use according to claim 1 wherein the composition includes PC in an amount of about 30% of total phospholipids.

11. The use according to claim 1 wherein the composition includes PS in an amount of about 15% of total phospholipids.

12. The use according to claim 1 wherein the composition includes PI in an amount of about 10% of total phospholipids.

13. The use according to claim 1 wherein the composition includes sphingomyelin in an amount of about 25% of total phospholipids.

14. The use according to claim 1 wherein the composition includes a non lipid component in an amount of about 45% by weight of the composition.

15. The use according to claim 1 wherein the non lipid component includes protein in an amount of about 25% by weight of the non lipid component.

16. The use according to claim 1 wherein the phospholipid is obtained or derived from bovine milk, or from a fraction, extract or product thereof.

17. The use according to claim 1 wherein the composition is provided as a unit dosage form such as a capsule, pill, caplet or like product, each unit dosage form having an amount of phospholipid from about 0.5 g to 5 g.

18. The use according to claim 1 wherein the composition is provided in a unit dosage form for providing a total amount of phospholipid per day of about 5 g or less.

19. The use according to claim 1 wherein the disease or condition is hepatomegaly.

20. The use according to claim 1 wherein the disease or condition is associated with elevated aspartate aminotransferase (AST) and alanine aminotransferase (AL T) levels.

21. (canceled)

22. The use according to claim 1 wherein the composition forms a food or dietary supplement, a food or a pharmaceutical substance.
